(19) United States
(12) Patent Application Publication
(10) Pub. No.: US 2012/0259289 A1
(43) Pub. Date: Oct. 11, 2012

(54) SYRINGE SYSTEM FOR CONTROLLED DELIVERY OR REMOVAL OF MATERIAL

(75) Inventors: Bobby L. Byrnes, Carlsbad, CA (US); Robert K. Shanahan, Carlsbad, CA (US); Douglas Arm, Carlsbad, CA (US); Jonathan Costabile, San Diego, CA (US); Jose F. Curiel, San Diego, CA (US)

(73) Assignee: CYTORI THERAPEUTICS, INC., San Diego, CA (US)

(21) Appl. No.: 13/450,262

(22) Filed: Apr. 18, 2012

Related U.S. Application Data
(63) Continuation of application No. 12/766,768, filed on Apr. 23, 2010, now abandoned.
Application No. 12/766,768 is a continuation of application No. PCT/US2008/081012, filed on Oct. 23, 2008.
(60) Provisional application No. 60/987,738, filed on Nov. 13, 2007.
(60) Provisional application No. 60/983,054, filed on Oct. 26, 2007.

(51) Int. Cl. A61M 5/315

(52) U.S. Cl. 604/227

(57) ABSTRACT

Embodiments disclosed herein relate to syringe systems for delivering and removing materials, instrument kits that include the syringe systems provided herein, and methods of using the syringe systems and kits provided herein.

### CROSS-REFERENCE TO RELATED APPLICATIONS

The present application is a continuation of U.S. patent application Ser. No. 12/766,768, filed Apr. 23, 2010, entitled “SYRINGE SYSTEM FOR CONTROLLED DELIVERY OR REMOVAL OF MATERIAL,” which is a continuation of International Patent Application No. PCT/US2008/081012, filed Oct. 23, 2008, by Byrnes et al., entitled “SYRINGE SYSTEM FOR CONTROLLED DELIVERY OR REMOVAL OF MATERIAL,” which claims priority to U.S. Provisional Application Ser. No. 60/983,054, filed on Oct. 26, 2007, by Byrnes, et al. and entitled “SYRINGE SYSTEMS FOR CONTROLLED DELIVERY OR REMOVAL OF MATERIAL,” and U.S. Provisional Application No. 60/987,738, filed on Nov. 13, 2007, by Byrnes, et al., and entitled “SYRINGE SYSTEMS FOR CONTROLLED DELIVERY OR REMOVAL OF MATERIAL.” The contents of all the aforementioned applications are expressly incorporated herein by this reference.

### FIELD OF THE INVENTION

The present invention relates to the field of medical devices, specifically to syringe devices for delivering and removing materials, e.g., cosmetic fillers and restorative agents including adipose tissue and adipose-tissue-derived materials, in reconstructive, cosmetic, or other procedures.

### BACKGROUND OF THE INVENTION

Instruments for injecting restorative agents in cosmetic and reconstructive procedures, for example, breast augmentation for restoration of tissue lost to lumpectomy or partial mastectomy, must provide ease of use in the controlled delivery of many small injections of restorative agents. Autologous tissue transplantation generally involves removal of a patient's adipose tissue from one site and subsequent reinjection at the defect site after some degree of tissue processing. The injection of many small, evenly distributed, droplets, as opposed to a few large ones, has proven superior for long-term tissue survival and regeneration. Since filling a typical lumpectomy or mastectomy defect can require on the order of 10 ml to more than 200 ml of adipose tissue, when restorative agent is delivered in e.g., 25-100 μl droplets, the surgeon can be required to make thousands of injections in a single procedure.

There is thus a need for an instrument that allows fine control in placing small amounts of material, is comfortable to use for long periods of time, and is capable of holding substantial volume.

### SUMMARY OF THE INVENTION

The present invention relates to a syringe delivery system that is flexible in the volume it can hold, allows a high degree of control in placing very small amounts of material, and maximizes operator comfort. The system comprises a thumbwheel-driven device that can be used interchangeably with disposable syringes, syringe components, cannulas and needles of various sizes to allow comfortably delivery or removal of materials, e.g., cosmetic fillers and restorative agents including adipose tissue and adipose-tissue-derived products such as adipose-derived stem, progenitor and other regenerative cells, in reconstructive, cosmetic, and other procedures. An adaptor fitting that allows use of disposable syringe barrels eliminates the need for a syringe barrel holder. This results in a more easily manipulated, more easily manufactured tool.

In certain embodiments, the present invention relates to a system for delivering material, comprising: (1) a hollow handle; (2) a syringe plunger assembly; (3) a syringe barrel; wherein said syringe plunger is held substantially within the handle when the syringe barrel is filled; (4) a driving mechanism comprising a thumbwheel portion and a gear portion, wherein said driving mechanism actuates the syringe plunger through a driving system, and; (5) an adaptor fitting for attaching the syringe barrel to the handle, wherein said adaptor fitting prevents longitudinal motion of the syringe barrel by securing the flanges of the syringe barrel. Said syringe system allows an operator to deliver multiple microdroplets of material or fluid placed within the syringe barrel to separate areas of tissue in a subject during a single procedure, or to extract material or fluid from a subject during a procedure.

In other embodiments, the present invention relates to a system for delivering material, comprising: (1) a syringe barrel; wherein the syringe barrel can contain the material to be delivered or the material to be extracted; and wherein the syringe barrel is comprised of a luer lock or a cap at one end to prevent the material from exiting the barrel; (2) a syringe plunger assembly with a full length hole in the center of the plunger; (3) a driving mechanism comprising a thumbwheel portion and a gear portion, wherein said driving mechanism actuates the syringe plunger through a driving system such that the material to be delivered travels through the full length hole in the plunger assembly to the delivery site via a needle or cannula attached to one end of the thumbwheel device or the one end of the plunger assembly; and optionally (4) an adaptor fitting for attaching the syringe barrel with the syringe plunger to the thumbwheel portion and gear portion, wherein said adaptor fitting prevents longitudinal motion of the syringe barrel by securing the flanges of the syringe barrel. In said syringe system embodiments, the syringe barrel functions as a handle thereby providing greater proximity to and greater control of the delivery needle or cannula to more accurately deliver multiple microdroplets of said fluid to separate areas of tissue in a subject during a single procedure, or to extract said fluid from a subject during a procedure. Furthermore, each turn of the thumbwheel mechanism allows a discreet “pearl” of tissue to be delivered.

In embodiments of the fluid handling system of the invention, said fluid is a filler or restorative agent for use in a reconstructive or cosmetic procedure. In further embodiments, said filler or restorative agent is a composition comprising one or more of the following: adipose-derived regenerative cells, adipose tissue, adipocytes, an injectable collagen, a hyaluronic acid filler, a botulinum toxin, a poly-L-lactic acid filler, a calcium hydroxyapatite filler, a silicone filler, and a microparticle filler. In specific embodiments, the fluid is a botulinum toxin.

In embodiments, the volume of each microdroplet is about 20 μl to 50 μl, 50 μl to 100 μl, 100 μl to 200 μl, 200 μl to 300 μl, 300 μl to 400 μl, or 400 μl to 500 μl. In various embodiments, the number of injections required for the procedure is about 5 to 10, 10 to 50, 50 to 100, 100 to 200, 200 to 300, 300 to 400, 400 to 500, 500 to 750, 750 to 1000, 1000 to 1500, 1500 to 2000, 2000 to 3000, 3000 to 4000, or 4000 to 5000.

In certain embodiments, systems of the invention can be used for either dispensing or extracting material. In related embodiments, the system can be used for both dispensing and extracting material.

In embodiments, the syringe plunger shaft, base and seal are disposable. In other embodiments, the syringe plunger base and seal are disposable and the syringe plunger shaft is reusable. The syringe barrel can be disposable, or part of a standard disposable syringe.

In embodiments of the invention, the system can interchangeably accommodate syringe barrels of more than one size. It can also interchangeably accommodate handles, needles and cannulas of more than one size.

In certain embodiments of the present invention the driving system comprises a rack and pinion mechanism.

In embodiments, depression of the thumbwheel allows release of the driving system, for example a driving system comprising a rack and pinion mechanism. In related embodiments, release of the thumbwheel allows the rack to move in reverse by a set amount.

In yet other embodiments of the invention, a sensor is used to detect pressure or vacuum in the syringe barrel. A user alarm can be activated when said pressure or vacuum detected exceeds an acceptable level, for example, an acceptable pressure level can be 1 atm, and an acceptable vacuum level can be −0.5 atm (gauge).

In embodiments of the invention, said reconstructive or cosmetic procedure is selected from the group consisting of: tissue defect filling; breast augmentation; lip augmentation; wrinkle filling, and; filling of defects caused by injury, trauma, infection, or surgery.

In specific embodiments, the system of the invention can accommodate a disposable 1 milliliter syringe barrel, or both plunger and barrel of a 1 milliliter syringe, and/or a 5 milliliter disposable syringe barrel or plunger and barrel, and/or a 10 milliliter disposable syringe barrel or plunger and barrel, and/or a 20 milliliter disposable syringe barrel or plunger and barrel, and/or a 60 milliliter disposable syringe barrel or plunger and barrel, and/or a 100 milliliter disposable syringe barrel or plunger and barrel, and/or a 140 milliliter disposable syringe barrel or plunger and barrel.

The invention also relates to a method for delivering microdroplets of a filler or restorative agent to a patient, or extracting microdroplets of a filler or restorative agent from a patient in a reconstructive or cosmetic procedure comprising using a system for handling a fluid that comprises: a hollow handle; a syringe plunger assembly; a syringe barrel; a driving mechanism comprising a thumbwheel portion and a gear portion, wherein said driving mechanism actuates the syringe plunger through a rack and pinion arrangement, and; an adaptor fitting for attaching the syringe barrel to the handle, wherein said adaptor fitting prevents longitudinal motion of the syringe barrel by securing the flanges of the syringe barrel; wherein said syringe plunger is held substantially within the handle when the syringe barrel is filled, and further wherein said system allows an operator to deliver multiple microdroplets of said fluid to separate areas of tissue in a subject during a single procedure, or to extract said fluid from a subject during a procedure.

In embodiments of the methods for delivering microdroplets of a filler or restorative agent of the invention, the fluid is a filler or restorative agent for use in a reconstructive or cosmetic procedure. In related embodiments, said filler or restorative agent is a composition comprising one or more of the following: adipose-derived regenerative cells, adipose tissue, adipocytes, an injectable collagen, a hyaluronic acid filler, a botulinum toxin, a poly-L-lactic acid filler, a calcium hydroxyapatite filler, a silicone filler, and a microparticle filler.

In certain embodiments of the methods of the invention, the volume of each microdroplet is about 20 μl to 50 μl, 50 μl to 100 μl, 100 μl to 200 μl, 200 μl to 300 μl, 300 μl to 400 μl, or 400 μl to 500 μl. In further embodiments, about 5 to 1000 injections are required for the procedure.

In other embodiments of the methods of the invention, the reconstructive or cosmetic procedure is selected from the group consisting of: tissue defect filling; breast augmentation; lip augmentation; wrinkle filling, and; filling of defects caused by injury, trauma, infection, or surgery.

In certain embodiments, the syringe barrel used was part a 1 ml, 5 ml, or 10 ml disposable syringe.

The invention also relates to kits for use in a reconstructive or cosmetic procedures, and other procedures including a procedure in which botulinim toxin is administered to a patient, comprising a system for handling a fluid, said system comprising: a hollow handle; a syringe plunger assembly; a syringe barrel; a driving mechanism comprising a thumbwheel portion and a gear portion, wherein said driving mechanism actuates the syringe plunger through a rack and pinion arrangement; and, an adaptor fitting for attaching the syringe barrel to the handle, wherein said adaptor fitting prevents longitudinal motion of the syringe barrel by securing the flanges of the syringe barrel; wherein said syringe plunger is held substantially within the handle when the syringe barrel is filled, and further wherein said system allows an operator to deliver multiple microdroplets of said fluid to separate areas of tissue in a subject during a single procedure, or to extract said fluid from a subject during a procedure.

In embodiments of the kit of the invention, the kit further comprises at least one syringe barrel useable with the syringe delivery system. In certain embodiments, the system for handling a fluid can interchangeably accommodate syringe barrels of more than one size. In yet other embodiments, the kit further comprises syringe barrels of more than one size useable with the syringe delivery system.

### BRIEF DESCRIPTION OF THE DRAWINGS

FIG. 1 An Assembled System of the Invention.

FIG. 2 Partially Exploded View of a System of the Invention.

FIG. 3 Cross-Sectional Partially Exploded View of a System of the Invention.

FIG. 4 Plunger Shaft. An example of a plunger shaft for use in the system of the invention. A. Shaft side view. Circle indicates area of detail shown in FIG. 4B. B. Shaft tooth detail. C. Base cross-sectional side view. Circle indicates area of detail shown in FIG. 4E. D. Bottom view. E. Base cross-sectional side view detail.

FIG. 5 Syringe Adaptor. An example of an adaptor for use in the system of the invention. A. Top iso view. B. Cross-sectional side view—1. Circle indicates area of detail shown in FIG. 5H. C. Cross-sectional side view—2. Hole for pin is shown. D. Side view—1. Hole for pin is shown. E. Side view—2. Hole for pin is shown. F. Bottom iso view. Hole for pin is shown. G. Bottom view. H. Pin detail.

FIG. 6 An Assembled System of the Invention.

### DETAILED DESCRIPTION OF THE PREFERRED EMBODIMENT

The syringe systems and methods of the present invention comprise a thumbwheel device that allows controlled delivery and/or removal of substantial volumes of fluid in small increments. It can interchangeably accommodate standard disposable syringes, including standard syringe components such as syringe barrels and standard or custom syringe plungers of different sizes. It can also interchangeably accommodate standard or custom needles and cannulas of different sizes. The syringe system is comprised of an adaptor fitting mounted onto the thumbwheel device that exploits the flanges of the syringe barrel to hold the syringe barrel in position during operation of the syringe delivery system via the thumbwheel device. This eliminates the need for an unwieldy syringe barrel holder and results in a smaller, more easily manipulated, more easily manufactured tool. Overall, the syringe systems of the present invention allow the user a greater level of dosing control, comfort and range of motion.

These features make the invention particularly useful for administering cosmetic fillers or restorative agents, e.g., autologous fat and/or fat-derived cells, in delicate reconstructive or cosmetic procedures, and for administering agents such as botulinum toxin in treatment of neuromuscular disorders, where drop-by-drop delivery of total volumes, ranging from less than 1 ml to over 100 ml and even more than 200 or 300 ml, are made over a period often exceeding an hour, two hours, or longer.

Accordingly, one preferred use of the present invention is in the field of cosmetic plastic surgery wherein the syringe system is used for augmentation in the dermis or subdermis to treat skin contour deficiencies caused by various conditions, including aging, environmental exposure, weight loss, child bearing, surgery, disease such as acne and cancer, or combinations thereof, or for beauty enhancement. The tissue augmentation method of the present invention is particularly suitable for treating frown lines, worry lines, wrinkles, crow's feet, facial scars, or marionette lines, or to augment facial features such as the lips, cheeks, chin, nose or under the eyes. Treatment of a patient may consist solely of using the syringe system of the present invention, or the syringe system may be used as part of additional cosmetic surgery such as a face or brow lift. As set forth herein, the syringe system may also be used for breast augmentation and breast reconstruction, and regions of the body that need tissue volume enlargement during reconstructive plastic surgery, such as after trauma or tumor resection.

The syringe system of the present invention may also be used in a variety of other circumstances where tissue volume augmentation is required. For example: in gastroenterology, wherein increasing the volume of tissue at the gastro-esophageal junction can be used to treat gastro-esophageal reflux disease, and increasing the thickness of the gastric mucosa to decrease the volume of the stomach to treat morbid obesity; in urology, where placing filler radially around the urethra at the neck of the urinary bladder can ameliorate incontinence; and in cardiology, whereby tissue filler may be placed in the ventricular wall to decrease the volume of the left ventricular chamber to treat heart failure, or in the pericardial space to place pressure on the outside of the heart, also intended to decrease the volume of the heart chambers and thereby treat heart failure; and in other applications well known to those skilled in the art.

In any of these clinical applications, the syringe system may be combined with any number of therapeutic biological materials, e.g., adipose derived stem, progenitor or other regenerative cells, or other bioactive substances which may be released from the biological materials over time, or be injected concurrently. The material delivered by the syringe system of the present invention can be any of a number of substances and may be of various physical states or combinations thereof, such as a non-viscous liquid, a viscous liquid, a gel, a powder, beads, flakes, continuous or discontinuous fibers, coils, fiber balls or mixtures thereof.

The syringe system of the present invention may be provided in a kit which includes the material to be delivered. Or the kit may consist solely of one or more syringe systems and the surgeon or operator provides the material to be delivered from an alternate source, e.g., in real time from the patient for autologous delivery or from a bank of cryopreserved autologous or allogeneic tissue or cells.

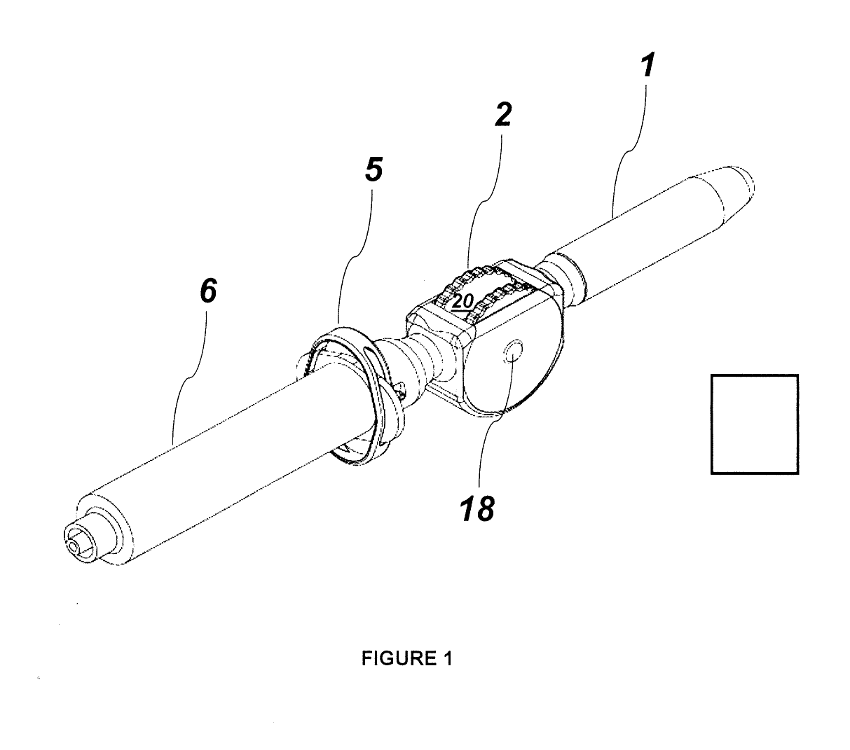

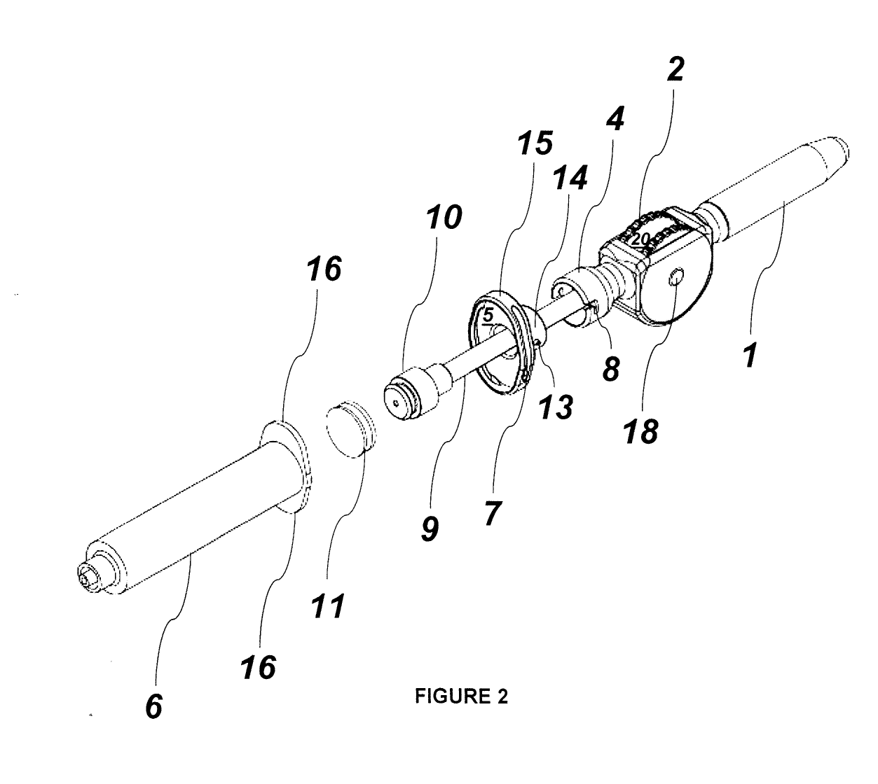

Examples of a syringe system of the present invention are shown in FIGS. 1, 2 and 6. In FIGS. 1 and 2, the barrel of a disposable syringe 6 can be fitted with a cannula as desired and evacuated or filled by moving a syringe plunger assembly, which includes a plunger base 10, a plunger seal 11, and a dentate plunger shaft 9, longitudinally within the barrel. The syringe plunger is actuated by a thumbwheel assembly 2 comprising a thumbwheel 20, a dentate pinion 3 which communicates with the dentate plunger shaft 9 through a rack and pinion arrangement, and an axle 18 that is situated crosswise through the handle. The syringe barrel is attached to the body of the dispenser using an adaptor fitting 5, which has slots 7 that grip the syringe barrel flanges and hold the syringe barrel in place. The hollow handle 1 accommodates the rear portion of the syringe plunger, which moves into the handle as the syringe is filled.

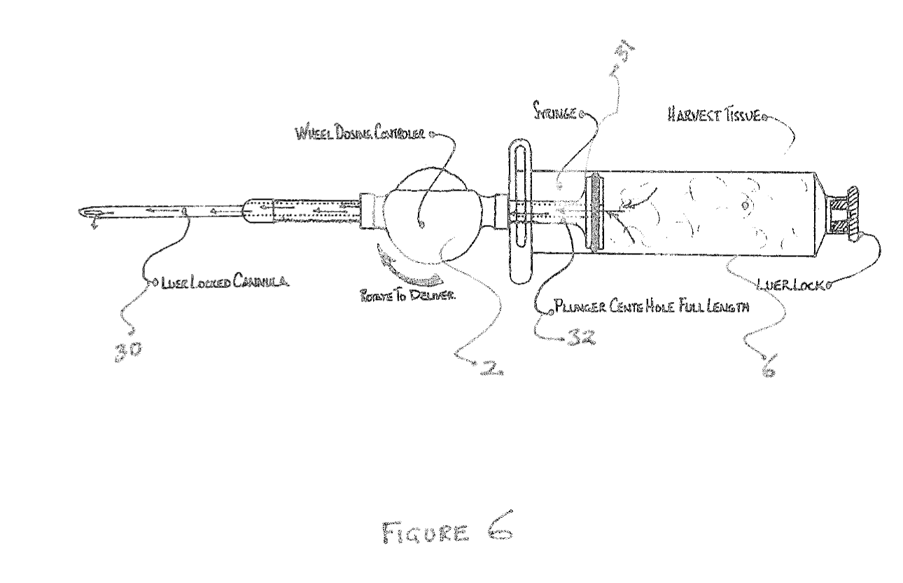

In FIG. 6, the barrel of a disposable syringe 6 can be filled by moving a syringe plunger assembly 31, which includes a dentate plunger shaft 9, longitudinally within the barrel wherein the plunger shaft has a hole 32 running through the center of the entire length of the plunger shaft. The plunger assembly 31 is actuated by a thumbwheel assembly 2 comprising a thumbwheel and a dentate pinion which communicates with the dentate plunger shaft 9 through a rack and pinion arrangement, and an axle that is situated crosswise through the handle. The syringe barrel may be attached to the body of the dispenser using an adaptor fitting which has slots that grip the syringe barrel flanges and hold the syringe barrel in place. The syringe barrel 6 serves as the syringe system handle and accommodates the material to be delivered. As the plunger assembly 31 is actuated by the thumbwheel assembly 2, the material to be delivered is forced from the syringe barrel 6, through center hole 32 running through the plunger shaft 31 into the needle or cannula 30 attached to the plunger assembly and onto the desired location in the form of a discrete “pearl” of material. Repetitive, controlled delivery is possible to achieve delivery of a “string of pearls” of desired material.

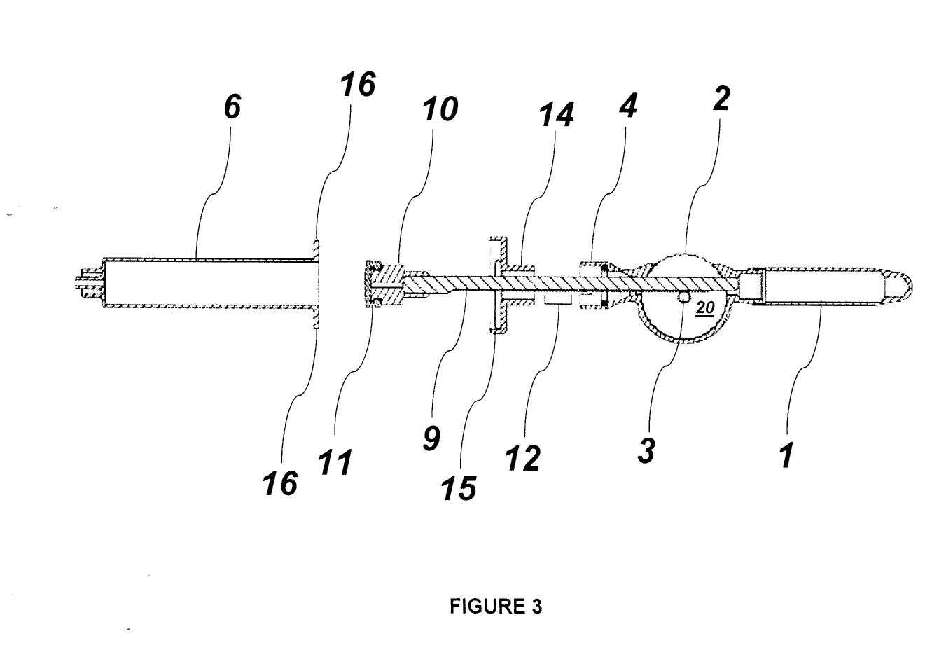

FIG. 3 is a longitudinal cross-sectional view of the system shown in FIGS. 1 and 2. This view reveals the rack and pinion arrangement comprising plunger shaft (rack) 12 and thumbwheel assembly gear (pinion) 3. The thumbwheel assembly gear (pinion) 3 can also be used in the syringe system shown in FIG. 6. In FIGS. 1 and 2, the thumbwheel assembly is situated within the handle 1 of the dispenser, and at least a portion of both thumbwheels 20 of the thumbwheel assembly are externally accessible in a position convenient for rotation by a user holding the dispenser. The handle accommodates the plunger shaft 12, while the plunger base 10 with a rubber seal 11 extends forward from the front end of the plunger shaft 9.

In an embodiment, a thread rod assembly is used instead of a rack and pinion assembly as a driving system for the plunger assembly. In this embodiment, the wheel turns from right-to-left or left-to-right, i.e., perpendicular to the plunger shaft, rather than from front-to-back or back-to-front, i.e., parallel to the plunger shaft. In further embodiments, an electric motor is used to turn the thread rod wheel of the thread rod assembly.

### Adaptor Fitting

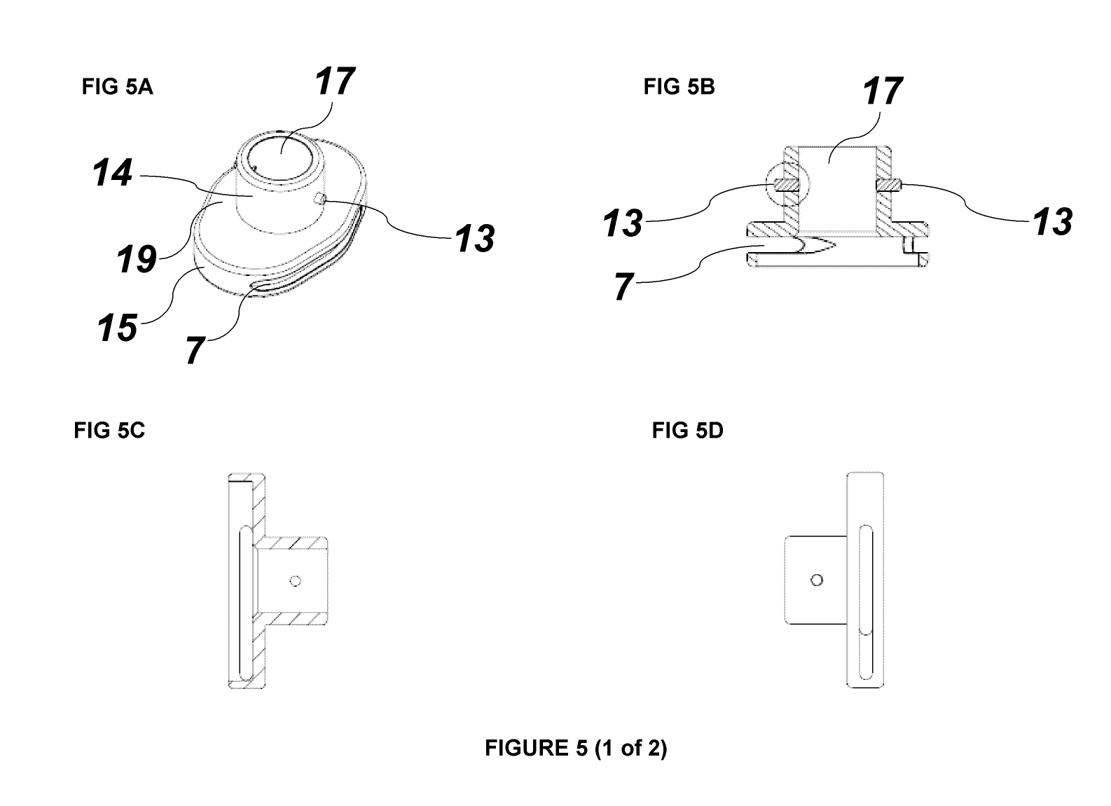

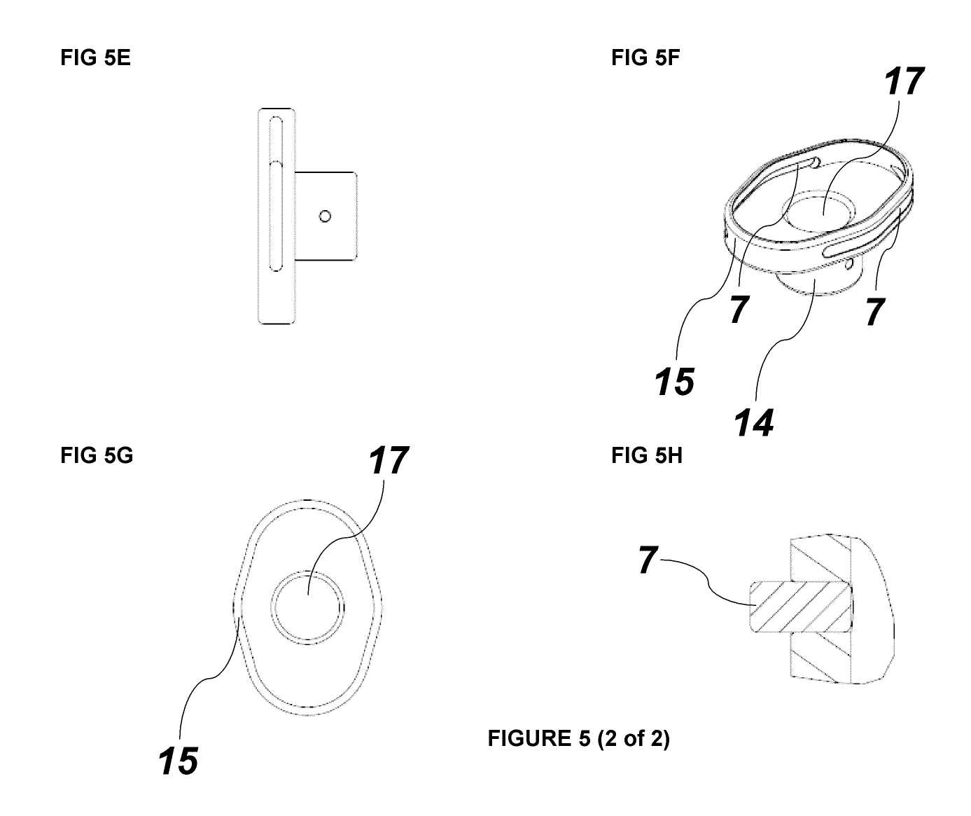

A syringe adaptor of the invention is shown in detail in FIG. 5. FIG. 5A provides an iso view from above the exemplary adaptor 5, i.e., the figure shows the part of the adaptor that faces the syringe dispenser when the adaptor is attached to the dispenser.

The adaptor fitting exploits the flanges of the syringe barrel to hold the syringe barrel in position during operation of the syringe delivery system. This eliminates the need for an unwieldy syringe barrel holder and results in a smaller, more easily manipulated, more easily manufactured tool.

The upper, cylindrical portion 14 of the adaptor has two pins 13 situated opposite one another. The pins protrude from the wall of adaptor portion 14 at a 90 degree angle. To attach the adaptor to the handle 1, portion 14 is first inserted into portion 4 of the handle. The pins track the slotted J-shaped grooves 8 of the handle assembly 1. After insertion into the handle fitting 4, the adaptor is rotated clockwise to lock the pins in place. The plunger shaft passes through the adaptor passage 17, and into the hollow handle, where its dentations mesh with those of the thumbwheel assembly gear in a rack and pinion arrangement.

FIG. 5B provides an iso view from below the adaptor 5, i.e., showing the part of the adaptor that faces the disposable syringe barrel when the adaptor is attached to the syringe.

The lower portion 15 of the adaptor has two grooves 7 on its vertical edge, one for engaging each of the flanges 16 of a disposable syringe barrel. To attach the syringe barrel to the adaptor 5, the flanged top of the syringe is fitted into the accommodating recessed area of the lower portion 15 of the adaptor, and the syringe barrel is turned 90 degrees on its longitudinal axis to securely lock the flanges 16 into the adaptor grooves 7.

In embodiments, the adaptor is made so that, when the adaptor is attached to the dispenser, the flanges are oriented so as to allow the operator to hold the dispenser close to the patient's body without interference from the flange. For example, if the surgeon holds the dispenser with the exposed portion of the thumbwheel facing away from the patient's body upright during use, it can be desirable that neither of the two flanges point toward the surface of the patient's body. The adaptor is made of stainless steel or another appropriate material that can be selected by one of skill in the art, and manufactured using methods known to those of skill in the art.

### Universal Adaptor

A self-adjusting adaptor fitting allows the same dispenser to be used interchangeably with syringes of different sizes. This universal adaptor fitting also uses the flanges of the syringe barrel to hold the barrel in place, and does not require a syringe barrel holder. In embodiments, this adaptor is comprised of two movable arms that curve inward toward each other. The arms are mounted opposite one another on the underside of the horizontal base plate 19 of the adaptor syringe fitting 15. In this embodiment, the adaptor syringe fitting does not require vertical portion 15 or grooves 7. The arms pivot in a plane that is parallel to the plane of the adaptor horizontal base plate 19. The pivot points for the arms are positioned depending on which way the arms will swing. For example, two curved (e.g., semicircular) arms can each be mounted with one arm end at each of two pivot points that are close together near or at the outer perimeter of the base plate. They can also be mounted with an end of each arm sharing a single pivot point. In these embodiments, the inner sides of the semicircular arms (on the concave side of the arm's curve) face one another and the arms pivot toward and away from each other to form a “claw” arrangement that clasps the syringe barrel beneath the flanges.

Alternatively, the two pivot points can be positioned opposite one another at or near the periphery of the base plate. In this embodiment, one end of each curved arm is mounted at each pivot point so that, again, the concave inner sides of the arms face each other. The arms swing out to allow positioning of the syringe barrel flanges and in to grasp the barrel beneath the flanges. These two planes are separated by a distance dictated by the height of the arm mountings, the distance being sufficient to accommodate the syringe flanges when the syringe is locked in place. The arms can use springs or another mechanism so that when they are in a “closed” position there is sufficient force to keep the syringe barrel in place by holding the flanges against the adaptor base plate. When closed the arms can partially or fully encircle the syringe barrel just beneath the syringe flanges, as long as the syringe barrel remains fixed to the handle.

In other embodiments, a self-adjusting adaptor has spring-loaded arms that can be mounted flush with or very close to the horizontal base plate, the arms having grooves that serve to hold the syringe flanges in the same manner as grooves 7.

Other ways of exploiting the syringe flanges to hold the syringe barrel in place can be used, for example, in other embodiments, a hinged or sliding clamshell mechanism, that either holds the flanges from their underside or within slots, is contemplated. Yet other methods for securing the syringe flanges can be determined by one of skill in the art using the teachings of the present invention.

The adaptor arms and mountings can be made from stainless steel or any other appropriate material when used in a reusable system. They can be made from a sterilizable plastic, e.g., polycarbonate, in a disposable dispenser.

### Thumbwheel Assembly

A thumbwheel assembly 2, as shown in FIGS. 1, 2 and 6, comprises a thumbwheel 20 and a gear or pinion 3 for driving the plunger, as well as an axle 18. The gear and plunger shaft shown are complementarily dentate or otherwise notched to allow the two to mesh and translate the gear's rotating motion into linear motion of the plunger. The exact configuration of the thumbwheel assembly can be selected based on ease of manufacturing, the needs of the user, etc. For example, in embodiments, the driving system could utilize a thumbwheel assembly wherein the thumbwheel gear is a friction wheel. In these embodiments, the gear and the plunger shaft have rough surfaces between which motion can be transferred.

In a specific embodiment, the thumbwheel assembly is comprised of two thumbwheels 20, shown as protruding from the top of the handle in FIGS. 1-3. These two thumbwheels flank a smaller dentate pinion 3 in the assembly and are joined by an axle 18. Because of the difference in the size of the thumb wheels and the pinion, the translation of the operator's movement of the larger wheels into movement of the smaller pinion results in very fine control of plunger thrust. In other contemplated configurations, the thumbwheel assembly has only one thumbwheel.

In embodiments, the thumbwheel assembly includes a quick-release mechanism so that when the thumbwheel is depressed by the user, the thumbwheel gear releases from the syringe plunger. This gives the plunger freedom to move in reverse, i.e., opposite the direction in which it is being driven, thereby lowering potentially excessive pressure or vacuum in the syringe barrel and possibly in the patient's tissue. Therefore, excessive pressure during delivery is relieved by allowing the plunger assembly to move away from the needle and/or patient, and excessive vacuum during extraction is relieved by allowing the plunger assembly to move toward the needle and/or patient.

The quick-release feature can be made, e.g., by setting each end of the thumbwheel axle 18 in a bearing block within a vertical track in the handle. A spring directly below the bearing block in each track exerts upward pressure on the axle, thereby maintaining the contact between the dentate thumbwheel gear and the dentate plunger shaft. When the user depresses the thumbwheel from above, opposing the spring's pressure, the thumbwheel gear lowers and its teeth disengage from the teeth of the plunger shaft.

When the quick-release mechanism is used to reduce pressure or vacuum in the syringe, the effect of depressing the thumbwheel can be adjusted according to the needs of the user by methods known to those of skill in the art. For example, reverse movement of the syringe plunger shaft when it is released from the thumbwheel gear can be slowed by a slip-regulating mechanism. This slip-regulating mechanism provides resistance to reverse motion of the plunger shaft. For example, a ratchet and pawl mechanism, wherein the teeth of the thumbwheel or the thumbwheel gear, or a second set of dentations in the plunger shaft serve as the ratchet, can be used. Other regulating mechanisms for providing resistance to reverse movement are known to those of skill in the art, for example, a protruding element can be installed opposite a series of depressions in the plunger shaft. The strength of the resistance to reverse movement of the plunger shaft will be influenced by the depth, shape, spacing, etc., of the protruding element and depressions. For example, in the use of a ratchet and pawl mechanism, the size, shape, and spacing of the ratchet teeth, and the size, shape and stiffness of the pawl, among other things, will affect the level of resistance provided. These and other parameters can be adjusted, using methods known to those of skill in the art, to calibrate the resistance so that when the thumbwheel is depressed one time, the plunger shaft will move in reverse by only a specified amount, for example one or more plunger indentations, before the rack and pinion mechanism reengages.

In embodiments, the resistance provided by the slip-regulating mechanism is adjusted or calibrated such that when the pressure or vacuum in the syringe barrel is excessive, then a reverse motion of the plunger is automatically triggered. This reverse motion continues until the pressure or vacuum level returns to an acceptable level. In embodiments, a toggled ratchet and pawl mechanism is used to regulate movement of the plunger shaft in one direction or the other, and therefore regulate either pressure or vacuum. In embodiments incorporating the thumbwheel quick-release, the spring action can be coordinated with the slip-regulating mechanism to achieve the desired amount of resistance.

It is understood by those of skill in the art that adjustment of the resistance provided by a slip-regulating mechanism can be made to take into account the force needed to move the specific rack and pinion or driver system used. Further, the engineering of the driver system can be coordinated with that of the slip-regulating mechanism to achieve the desired balance between the force to move each one.

In other embodiments, a cracking valve, or directional valve, is placed on the plunger assembly at the junction of the seal and the interior of the syringe barrel so that excessive pressure is relieved through the valve. In certain embodiments, pressure is relieved by allowing fluid from the syringe barrel to pass through the cracking valve and a lumen that extends through the plunger assembly. For example, a passage can be formed within the plunger base and shaft. Fluid expelled through this passage can, e.g., empty into the handle of the fluid handling system. In certain embodiments, a window in the handle allows the operator to see expelled fluid, prompting him to decrease pressure by turning the thumbwheel more slowly, not turning the thumbwheel at all, or by using the quick-release mechanism to allow reversal of plunger movement. Use and placement of cracking valves is known to those of skill in the art, and cracking valves are commercially available.

Caution regarding excessive pressure is particularly important during delivery of cosmetic fillers or restorative agents to the face, where arteriolar microembolism presumably due to excessive delivery pressure in autologous fat transfer, has been known to injure patients and even result in fatality (Butterwick, et al., 2007, “Autologous Fat Transfer: An In-Depth Look at Varying Concepts and Techniques,” Facial Plast. Surg. Clin. N. Am. 15: 99-111, and Allah, et al., 2006, “Multiple embolizations of the branches of the ophthalmic artery: an unknown serious complication of facial surgeries,” J Fr Ophtalmol. 29(1):51-7). It has been recommended that pressure during delivery not be allowed to increase above 1 atm (gauge).

In adipose tissue extraction, cell viability has been reported to decrease when fat is removed under vacuum levels of −700 mmHg (about −0.92 atm) but not when vacuum levels are lower (Butterwick, et al., 2007, and Shiffman, et al., 2001, “Fat Transfer Techniques: the Effect of Harvest and Transfer Methods on Adipocyte Viability and Review of the Literature,” Dermatol. Surg.; 27(9):819-26.)

In certain embodiments, depression of a quick-release thumbwheel allows rapid filling of the syringe barrel with cosmetic filler or restorative agent. This prevents the user from having to completely or partially disassemble and reassemble the system when the syringe barrel is empty. In this embodiment, a vacuum line can enter the handle and assist the forward and backward motion of the plunger assembly, e.g., by pulling back on the plunger base and seal. Application of a vacuum to drive the plunger assembly in the context of present invention can be accomplished using methods known to those of skill in the art. The vacuum could also be activated by mechanisms, known to those of skill in the art, other than depression of a quick-release thumbwheel.

The thumbwheel assembly can be made from stainless steel or another appropriate material as selected by one of skill in the art, and manufactured using methods known to those of skill in the art. In embodiments, it can be calibrated and graduation marks made on the handle and thumbwheel to indicate volume administered or extracted.

### Syringe Plunger Assembly

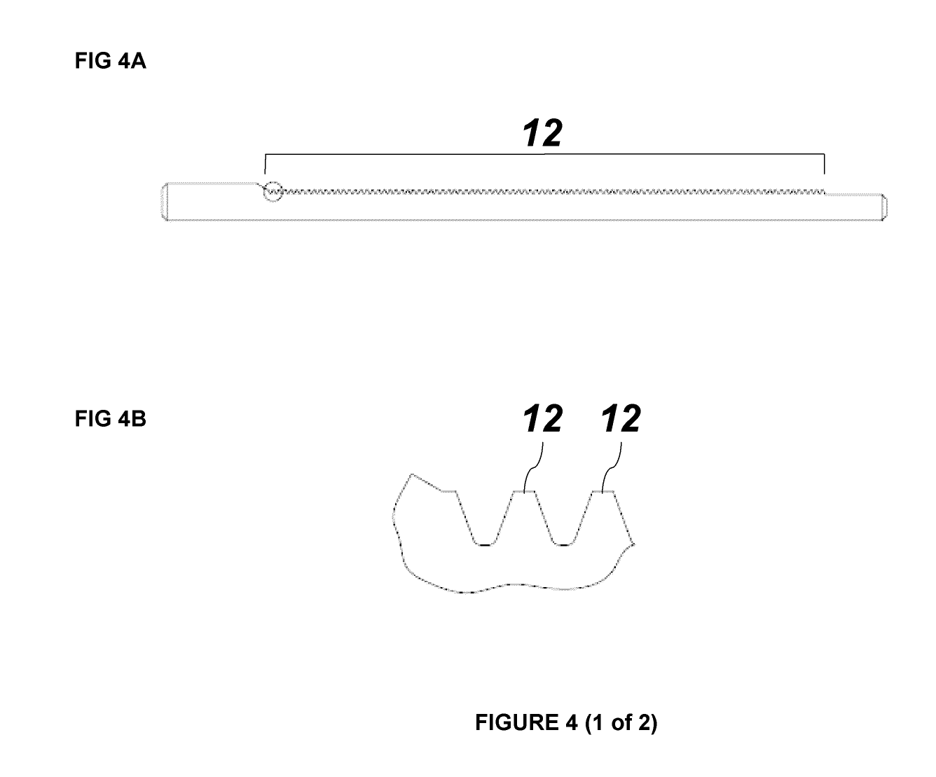

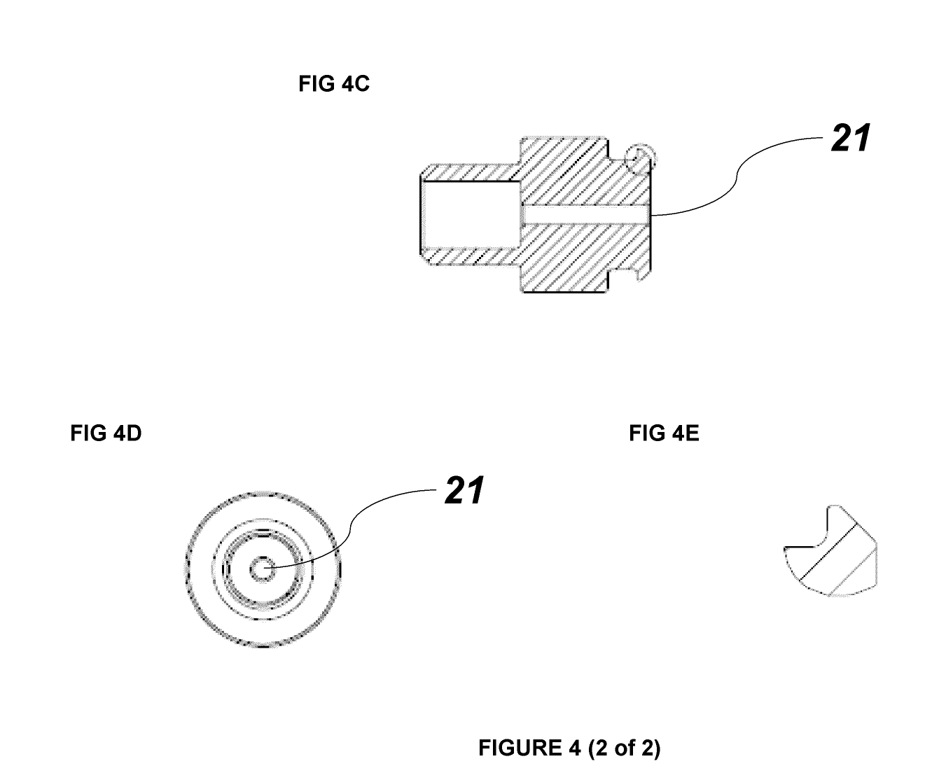

A syringe plunger assembly useful in the system and methods of the invention is shown in detail in FIG. 4. FIG. 4A shows the dentate plunger shaft (piston), and FIG. 4B shows detail of the plunger shaft teeth. The size and shape of the teeth are designed to mesh with those of the thumbwheel gear. The plunger as shown must be inserted into the dispenser handle with the dentate side oriented downward, to ensure that the teeth mesh with the teeth on the thumbwheel gear below. The operator can roll the thumbwheel to draw the plunger shaft back into the handle. In embodiments, the plunger has a shape (e.g., flat or grooved longitudinally on one side) that is complementary to the shape of the orifice in the adaptor and/or the handle (in front of the thumbwheel gear), to allow insertion in only the correct orientation (with the teeth of the plunger shaft facing the teeth of the thumbwheel gear).

FIG. 4C shows a side view of the plunger base in cross-section. The plunger base (10 in FIG. 2) has a recessed area for accepting the plunger shaft, a vent 21 to allow air to vent when the shaft is inserted, and a groove behind the tip for fitting on the seal, shown in FIG. 4D and also as 11 in FIG. 2. There are numerous methods, known to those of skill in the art, of designing and constructing the shaft, base, and seal so that they fit together and within the syringe barrel appropriately and function properly in the context of the present invention. In embodiments, the plunger shaft and base can be made as a single part, rather than as separate parts that are later attached to one another. This combined part can be reusable or disposable.

The plunger assembly parts can be made of any appropriate material that can be selected by one of skill in the art. For example, the plunger shaft and base can be made from stainless steel, or a plastic such as polycarbonate. The plunger seal can be made of rubber or any appropriate substance that does not react with the material in the syringe.

The shaft, base and seal can be manufactured using methods known to those of skill in the art and as desired by the user. Selection of materials for making any part of the system will depend in part on whether the part will be reusable or disposable. It is preferred that reusable parts be autoclavable and disposable parts sterilizable. The material selected for the plunger shaft should have appropriate qualities (e.g., strength, texture, etc.) so that the rack and pinion mechanism is robust and does not slip unintentionally. The plunger shaft can be reusable or disposable. A disposable plunger shaft made from, e.g., polycarbonate, can be combined with a disposable plunger base and plunger seal. In embodiments, a reusable plunger shaft made from, e.g., stainless steel, can be combined with different disposable bases and seals depending on the size of the syringe to be used.

In embodiments of the present invention, the entire fluid handling system can be a disposable device that is not intended for reuse. In accordance with other embodiments of the present invention, the system can be resterilized, to enable reuse. In accordance with still other embodiments of the present invention, components of the system are reusable, while other components are not.

### Actuator Mechanisms

In embodiments of the present invention, linear movement of the plunger is actuated by rotation of the thumbwheel assembly gear. The thumbwheel assembly gear is actuated by rotating the thumbwheel. In different embodiments, the thumbwheel itself can be actuated directly or indirectly by the operator. Actuation by the operator can be assisted by, e.g., a powered actuator. For example, the operator can supply power through a switch (e.g., push-button, on-off, adjustable, etc.) to a motor that in turn rotates the thumbwheel. Alternatively, a vacuum generator can be linked to the system for actuation as described herein.

Many potential variations for providing a powered actuation mechanism are available and known to those of skill in the art. In general, selection is made based on the requirements and preferences of the user. For example, mechanisms can be selected by considering their potential benefit (e.g., increased ease of use for a handicapped user) in relation to the additional size, bulk and weight of the mechanism.

### Handle

The handle of the dispenser, designed to be held by the user, has a fitting 4 to attach syringe adaptor 5 and hollow space within to accommodate plunger shaft 9. In embodiments, the handle is modular, wherein the portion of the handle to the rear of the thumbwheel can be removed and replaced with handles of different sizes depending on the needs of the user. For example, when a larger syringe with a longer plunger shaft is used, the shorter handle can be exchanged for a longer handle to accommodate the extra shaft length. Alternatively, the handle can have an open end. Handle size, shape, etc., can be selected based on user comfort, e.g., to suit the user's hand size or preference. The handle can be made of stainless steel or any other appropriate material selectable by one of skill in the art.

### Syringes

Syringes for use with the systems and methods of the present invention include standard disposable syringes, including, but not limited to, syringes that hold volumes of 1 ml, 2 ml, 3 ml, 5 ml, 10 ml, 25 ml, 50 ml, 60 ml, 100 ml, 140 ml, etc. In embodiments, the syringe has a luer lock tip. Standard disposable luer lock syringes are widely available from commercial sources, including Henke Sass Wolfe, GMBH (e.g., Norm-Ject and Soft-Ject syringes, distributed in North America by Air-Tite Products Co., Inc., Virginia Beach, Va.). Standard or nonstandard syringes of various sizes, including the corresponding plungers (shaft, base and seal), can be manufactured to suit a user's needs. Syringe plungers, as well as plunger bases and seals, can be disposable or reusable and are made according to methods known to those of skill in the art.

The needle or cannula can be selected by one of skill in the art based on various aspects of the specific procedure or treatment. The needle or cannula has a proximal end and a distal end, and a lumen extending from one end to the other. In one embodiment, the needle is 14-20 gauge. The syringe size can vary as discussed elsewhere herein, depending on the volume of fluid it will hold. Needles or cannulae of, e.g., 12-35 gauge or 1 to 6 mm, respectively, having different tip conformations, including blunt tip, sharp tip, Mercedes tip, birdcage, multiple hole, etc., and different lengths, are useful depending on the specific indication. A variety of needles and cannulae are readily available from commercial sources.

In specific embodiments, a 25 to 35 gauge needle is used for Botox injections. In other specific embodiments, a 1 ml, 10 ml, or 25 ml syringe is used for extraction of fat, with, e.g., a 2.0, 2.5, 3.0, 3.5, or 4.0 mm cannula, or a 12 to 25-gauge needle. Larger syringes, e.g., 50 ml, 60 ml, 100 ml, etc., as discussed elsewhere herein, are also used. In yet other embodiments, cosmetic or restorative materials, e.g., fat, autologous fat, or fat mixed with fat-derived cells, are delivered using 2 to 6 mm cannulae and 14 to 18-gauge needles.

Custom needles can be made to the user's specifications, e.g., from steel that is first heated until it is molten and then drawn through a die designed to meet the size requirements of the needle. As it moves along the production line, the steel is further formed and rolled into a continuous, hollow wire. The wire is appropriately cut to form the needle. Some needles are significantly more complex and are produced directly from a die casting. Other metal components on the needle are also produced in this manner.

Custom syringe barrels can be made by different methods depending on the needs of the user, e.g., whether the parts must be disposable, reusable, autoclavable, sterilizable, etc., and on the raw materials used. One method of production is extrusion molding. The plastic or glass is supplied as granules or powder and is fed into a large hopper. The extrusion process involves a large spiral screw, which forces the material through a heated chamber and makes it a thick, flowing mass. It is then forced through a die, producing a continuous tube that is cooled and cut.

For pieces that have more complex shapes, including the ends, the plungers, plunger base, safety caps, etc., injection molding can be used. In this process the plastic is heated, converting it into a liquid. It is then forcibly injected into a mold that is the inverse of the desired shape. After it cools, it solidifies and maintains its shape after the die is opened. The rubber plunger seal can also be manufactured by injection molding. Later, the seal is attached to the plunger base.

When all of the component pieces are available, final assembly can occur. For example, as the tubes travel down a conveyor, the plunger is inserted and held into place. The ends that cap the tube are affixed. Graduation markings may also be printed on the main tube body at this point in the manufacturing process. The machines that print these markings are specially calibrated to ensure they print measurements on accurately. Depending on the design, the needle or cannula can also be attached at this time, along with a safety cap.

After all of the components are in place and printing is complete, the syringes can be put into appropriate packaging. Since sterility of the device is imperative, steps are taken to ensure they are free from disease-causing agents. They are typically packaged individually in airtight plastic. Groups of syringes can be packed into boxes, stacked on pallets, and shipped as needed.

### Extraction Device

In embodiments, the system of the present invention can be used for fluid extraction or it can function as a combination delivery and extraction instrument (i.e., it is reversible). To facilitate the dispenser's use for either delivery or extraction, the slip-regulating mechanism, as previously described, can help prevent unintended reverse movement of the plunger shaft caused by vacuum pressure in the syringe barrel during extraction. This allows the user to control the pressure or vacuum range during use.

As described above, the pawl of a ratchet and pawl slip-regulating mechanism can engage either the wheel teeth or indentations in the plunger shaft. During extraction, this mechanism prevents unintentional reverse slipping of the plunger shaft and inadvertent release of extracted material. This action is balanced with the prevention of an overly strong vacuum in the syringe barrel, which can be unsafe. In a combination delivery and extraction instrument, the slip-regulating mechanism would be designed to provide the desired balance of resistance in either direction.

In embodiments, the slip-regulating mechanism comprises a ratchet and pawl that can be set to prevent slipping in either one direction or the other. The user can simply switch, or toggle, the setting depending on whether he intends to use the dispenser for delivery or extraction. Switching the direction in which a ratchet and pawl catches is described elsewhere herein and is a feature that is known to and that can be applied to the present invention by one of skill in the art.

Also as previously described the design of the slip-regulating mechanism, including the distance between and the depth of the indentations, can be varied depending on how the dispenser will be used. For example, the viscosity of the material being extracted and the size of syringe used will affect the resistance necessarily exerted by the slip-regulating mechanism. A slip-regulating mechanism can also be useful for metering purposes, in either an extraction or delivery instrument, e.g., movement of the pawl in a ratchet and pawl slip-regulating mechanism by a single indentation or predetermined number of indentations will represent a certain quantity of material extracted or delivered. When the user feels a periodic “click” as the slip-regulating mechanism moves, the number of clicks can indicate to the surgeon the progress of the procedure without requiring constant visual inspection of the instrument (e.g., to check the syringe volume) during use.

### Pressure and Vacuum Sensors

In embodiments, a pressure or vacuum sensor is installed in the syringe plunger to alert the user of unacceptable pressure or vacuum levels within the syringe barrel, to avoid harm to the patient. When the alarm is activated, the user can, e.g., use the thumbwheel quick-release to alleviate excess pressure or vacuum. The sensors can be designed to detect specific pressure or vacuum thresholds. Sensors useful in these embodiments, e.g., electronic sensors, are known in the art and commercially available. They can be incorporated according to methods known to those of skill in the art.

### Indications

The system and methods of the present invention are contemplated for use in reconstructive or cosmetic procedures including, but not limited to, filling tissue defects, e.g., breast defects caused by lumpectomy, partial mastectomy, mastectomy, wrinkles, pockmarks, defects due to injury, including burns, traumatic injuries such as facial bone fractures, congenital abnormalities such as cleft lip or cleft palate, developmental abnormalities, defects caused by infection or disease, or removal of cancers or tumors. Use of the system of the invention is also contemplated for removing tissue, e.g., adipose tissue, for use in reconstructive or cosmetic procedures, e.g., directly or after further processing.

The present invention is also contemplated for use in injecting botulinum toxin for treatment of muscle disorders and other conditions, which can require many small-volume injections. Botulinum toxin and its potential uses are described in, e.g., U.S. Pat. No. 7,211,261, “Stable liquid formulations of botulinum toxin,” incorporated herein by reference. Botulinum toxin, particularly botulinum toxin Type A, has also been shown to be an effective treatment of spastic muscle disorders. A single treatment regimen (which may include multiple intramuscular injections) can provide relief from uncontrollable muscle spasm for as long as several months. For example, BOTOX is approved by the U.S. Food and Drug Administration for localized injection into the ocular orbit for treatment of blepharospasm. Other indications include other focal dystonias, such as laryngeal dystonia, Meige's syndrome (oromandibular dystonia; orofacial dyskinesia), spasmodic torticollis, limb dystonia, animus, and urinary detrusor-sphincter dyssynergia, blepharospasm, strabismus, hemifacial spasm as well as rhinorrhea, otitis media, excessive salivation, asthma, spastic colitis, excessive stomach acid secretion (see, for example, U.S. Pat. No. 5,766,005), headache associated with migraine, vascular disturbances, neuralgia or neuropathy (U.S. Pat. No. 5,714,468; WO 95/3041), arthritis pain (WO 95/17904), disorders of the gastrointestinal tract involving striated or smooth muscle (U.S. Pat. No. 5,674,205), relaxation of the perineum during childbirth (U.S. Pat. No. 5,562,899), or relief of jaw-clenching (U.S. Pat. No. 5,298,019). Botulinum toxin Type A has been also injected locally to achieve cosmetic relief of muscle tone which causes “frown lines” on the face and to achieve a “browlift” and has been found to be useful when injected intracutaneously for treating focal hyperhydrosis (excessive sweating; WO 95/28171; U.S. Pat. No. 5,766,605) as well as for treating juvenile curvature of the spine (U.S. Pat. No. 5,053,005) adult and juvenile cerebral palsy (U.S. Pat. No. 5,298,019; WO 93/05800), and spasms and involuntary contractions caused by cerebral palsy, multiple sclerosis or Parkinson's disease (U.S. Pat. No. 5,183,462). These references are herein incorporated by reference in their entireties.

U.S. Pat. No. 7,208,166, “Use of botuline toxin to obtain a product to be used in articular pathologies, particularly coxarthrosis, epicondylitis and rotator muscle cap pathology,” incorporated herein by reference, reports the use of botulinum toxin to treat articular pathologies connected with muscular tensions and contractions, particularly coxarthrosis, or arthrosis of the hip, epicondylitis of the elbow, or rotator muscle cap pathology relating to the periarticular structures of the shoulder. The patent describes administering a botulinum toxin type A composition into a thigh muscle of a patient suffering from coxarthrosis, wherein said thigh muscle is selected from at least one member of the group consisting of the long adductor muscle, great adductor muscle, iliopsoas and tensor muscle of the fascia lata, wherein said composition comprises a mixture of botulinum toxin type A and a physiological solution containing between 0.45% and 1.0% sodium chloride, and wherein said endo-articular pressure exerted by said thigh muscle on the femoral head against the cotyl in the hip is reduced.

### Fillers and Restorative Agents

Cosmetic fillers and restorative agents useful with the systems and methods of the present invention are known in the art and commercially available. They include, but are not limited to: autologous and nonautologous adipose tissue and its individual components, e.g., adipocytes, adipose-derived regenerative cells and mixtures thereof; microparticle fillers (e.g., Artecoll, polymethylmethacrylate plastic beads suspended in bovine collagen, and Reviderm, microparticles in a hyaluronic acid base); hyaluronic acids (e.g., Restylane, a nonanimal stabilized hyaluronic acid, Perlane, Macrolane, DermaLive/DermaDeep, Juvederm); injectable collagens (e.g., CosmoDerm/CosmoPlast, Dermalogen, Zyderm, Zyplast); silicone fillers (e.g., Silikon 1000, Adatosil 5000, SilSkin); GoreTex, poly-L-lactic acid fillers; calcium hydroxyapatite fillers, and; botulinum toxin type A (as further described below). These and other fillers and restorative agents can be used as deemed safe for the patient and as appropriate for the indication at hand. Fillers and agents are selected to maximize safety and minimize adverse reactions. For example, for many fillers not derived from autologous tissue, it is of particular importance to assess patient tolerance using methods known to those of skill in the art.

Methods for isolating or producing these and other cosmetic fillers and restorative agents are well known in the art. For example, systems and methods for isolating adipose-derived regenerative cells and for using them as a restorative agent in reconstructive or cosmetic procedures are described in U.S. Patent App. Pub. No. 2005/084961, “Systems and methods for separating and concentrating regenerative cells from tissue,” and U.S. Patent App. No. 2005/0025755, “Methods of using adipose tissue-derived cells in augmenting autologous fat transfer,” both incorporated herein by reference.

Botulinum toxin is a polypeptide product of the anaerobic bacterium Clostridium botulinum. The toxin causes muscle paralysis in mammals by blocking presynaptic release of the neurotransmitter acetylcholine at the neuromuscular junction. There are at least eight known serologically distinct botulinum toxins (A, B, C1, C2, D, E, F and G), and the term can refer to as additional botulinum toxins having the same general ability to inhibit cholinergic neurotransmission, which form the active molecule. The properties of the various botulinum toxins are described by Jankovic and Brin, The New England Journal of Medicine, Vol. 324, No. 17, 1990, pp. 1186-1194, and by Charles L. Hatheway in Chapter 1 of the book entitled Botulinum Neurotoxin and Tetanus Toxin, L. L. Simpson, Ed., published by Academic Press Inc. of San Diego, Calif., 1989, the disclosures of which are incorporated herein by reference. Commercially available toxins include type A toxin, Dysport (IPSEN PHARMACEUTICALS Ltd., Dublin, Ireland), type A toxin, BOTOX®, (ALLERGAN Inc., Irvine, Calif., USA), and type B botulin toxin, MYOBLOC® (ELAN PHARMACEUTICALS INC., South San Francisco, Calif., USA).

Botox can also include a carrier protein that is also derived from C. botulinum and which complexes with the active molecule. Botulinum toxin serotypes are related pharmacologically but are immunologically distinguishable. Generally, the active toxin molecule has a molecular size of between about 145 and 170 kilodaltons (kD). In the context of the present invention, it is understood that the toxin protein includes toxins and carrier proteins that are isolated from natural sources, as well as corresponding toxins and carrier proteins that are produced recombinantly according to methods known in the art. Moreover, use of proteins having amino acid sequences that include conservative amino acid substitutions, including deletions, with respect to known botulinum toxin sequences, are also contemplated.

### Delivery Methods

Dosage and delivery methods commonly used for the treatments described herein make the fluid handling systems and methods of the present invention particularly useful with these treatments. For example, techniques for placement of autologous fat in cosmetic and reconstructive procedures include the “Coleman method” and FAMI (fat autograft muscle injection). Both of these methods require numerous small injections and can therefore be facilitated by use with the systems and methods of the present invention.

The Coleman method is described, e.g., by Butterwick, et al. (2007), as involving intricate layering of minute quantities of fat within multiple tissue planes not only in the subcutaneous plane but also adjacent to bone, fascia, and muscle. This report describes placement of each droplet (“microdroplet” or “parcel”) of fat within a distance of about 1.5 mm of living vascularized tissue. Typically, 30 or 40 passes are required to empty a 1-ml syringe with a blunt tipped 17-gauge or 18-gauge cannula. Quantities injected for a full face often exceed 100 ml. Thus, using the Coleman method, a 100 ml procedure would require 3000 to 4000 needle passes, each delivering about 20 to 50 microliters of fat.

The FAMI method, also described by Butterwick, et al. (2007), involves injecting fat into or immediately adjacent to the muscles of facial expression. According to Butterwick, et al., the FAMI technique involves placing volumes of about 20 to 70 ml using blunt-tipped cannulae. A 1 ml syringe is emptied in one to three passes. A 70 ml procedure would thus require up to 200 or more needle passes using FAMI.

It is contemplated that a total volume of fluid, ranging from less than 1 ml, e.g., 0.5 ml, to greater than 300 ml, is administered in a procedure using the methods of the invention. A procedure can involve, e.g., treating a tissue defect, treating multiple defects in one area of the body, or a procedure can be one in a series of treatments using the systems and methods of the present invention. Single injections can range in volume from 20 μl or less to several milliliters.

In embodiments, the system and methods of the present invention are used in combination with one or more other treatments, e.g., fat grafting, use of prosthetics, or administration of other compounds.

### Kits

The invention includes kits, e.g., instrument kits, for use in a cosmetic or restorative procedure, or a procedure in which botulinim toxin is administered to a patient. These kits comprise syringe delivery systems of the invention. These kits can further comprise one or more syringes and syringe plunger assemblies that can be accommodated by the syringe delivery systems of the invention, cannulae or needles useful in the methods of the invention, etc. In embodiments the kit can further include a syringe cap, so that a syringe filled using the methods of the invention can be capped and stored for later use. In embodiments, wherein a reusable plunger shaft is provided, the syringes are each provided with a disposable plunger and seal.

A syringe filled with fat extracted from a patient using the system and methods of the invention can be capped, or otherwise closed, and stored at ⁻20° C., ⁻80° C., or at another temperature as determined optimal by one of skill in the art. The fat can be stored as extracted (e.g., in tumescent fluid), or alternatively, preservatives (e.g., DMSO, trehalose, etc.), or regenerative cells, can be added. Fat storage for later use, including use for delivery to a patient using the methods of the present invention, is described by, e.g., Butterwick, et al. (2007).

In certain embodiments, one or more components of the syringe system are manufactured from materials that retain structural integrity and resistance to damage when stored at temperatures as low as approximately −150° C. (the temperature present within the vapor phase of a liquid nitrogen storage tank) or −196° C. (the temperature present within the liquid phase of a liquid nitrogen storage tank). In these embodiment the syringe body may be manufactured from the same materials used in other rigid cryostorage devices such as cryovials (polypropylene). Other materials such as polyolefins may also be suitable. If storage in the liquid nitrogen phase is performed it may be preferred to use an overwrap or other sealed outer container to prevent infiltration of liquid nitrogen into the body of the syringe as this may result in explosive damage during thawing due to rapid evaporation and expansion of trapped liquid nitrogen.

Citation of documents herein is not intended as an admission that any of the documents cited herein is pertinent prior art, or an admission that the cited documents are considered material to the patentability of the claims of the present application. All statements as to the date or representations as to the contents of these documents are based on the information available to the applicant and do not constitute any admission as to the correctness of the dates or contents of these documents.

The contents of all cited references, including literature references, issued patents, published patent applications, and co-pending patent applications, cited throughout this application are hereby expressly incorporated by reference in their entirety.

What is claimed is:

1. A syringe delivery system, comprising:
a hollow handle;
a syringe plunger assembly;
a syringe barrel comprising flanges;
a driving mechanism comprising a thumbwheel portion and a gear portion, wherein said driving mechanism actuates the syringe plunger through a driving system, and;
an adaptor fitting that attaches the syringe barrel to the handle, wherein said adaptor fitting prevents longitudinal motion of the syringe barrel by securing the flanges of the syringe barrel;
wherein said syringe plunger is held substantially within the handle when the syringe barrel is filled, and further wherein said system is configured to deliver multiple microdroplets of a fluid to separate areas of tissue in a subject during a single procedure, or to extract said fluid from a subject during a procedure, wherein said fluid comprises a filler or a restorative agent suitable for a reconstructive or a cosmetic procedure.
